(19) United States
(12) Patent Application Publication
(10) Pub. No.: US 2024/0198400 A1
(43) Pub. Date: Jun. 20, 2024

(54) METHOD AND CHEMICAL FORMULATION FOR TREATING CONTAMINATED MEDICAL WASTE

(71) Applicant: CENTRO DE RETINA MÉDICA Y QUIRÚRGICA, S.C., Zapopan, Jalisco (MX)

(72) Inventors: Arturo SANTOS GARCÍA, Zapopan, Jalisco (MX); Gisela Hialita SÁNCHEZ SOSA, Zapopan, Jalisco (MX)

(21) Appl. No.: 18/267,475

(22) PCT Filed: Dec. 15, 2021
(86) PCT No.: PCT/MX2021/050087

§ 371 Date: Feb. 9, 2024

(30) Foreign Application Priority Data
Dec. 16, 2020 (MX) MX/A/2020/013871

(51) Int. Cl. B09B 3/35; A01N 37/16; A01P 1/00; B09B 3/60; B09B 101/65

(52) U.S. Cl. CPC B09B 3/35; A01N 37/16; A01P 1/00; B09B 3/60; B09B 2101/65

(57) ABSTRACT

Method and chemical formulation for treating contaminated medical waste by means of grinding and disinfection with peracetic acid. The invention relates to a peracetic-acid-based formulation and a continuous in-situ method for grinding and disinfecting, with the peracetic acid formulation, medical waste contaminated with viruses, bacteria, etc.

### FIELD OF THE INVENTION

The present invention pertains to the field of biotechnology, environment, and health. The invention consists of a formulation and method used for the sterilization, transformation, and final disposal or recycling of inputs, in particular personal protective equipment (PPE) exposed to SARS-CoV-2 (SARS: Severe Acute Respiratory Syndrome) that is generated in health care centers, medical offices, residential homes, industry, educational institutions, recreation centers, and public places.

### BACKGROUND OF THE INVENTION

As the COVID-19 pandemic spreads around the world, there is growing concern about how to manage the waste generated by suspected infected patients, by the staff caring for them, and by clinical laboratories that perform diagnostic tests. In establishments such as supermarkets, restaurants, factories, educational institutions, recreation centers, and public places, personal protective equipment is used both by the personnel providing the service and by the users and is periodically discarded and is potentially contaminated with SARS-CoV-2. In addition, people diagnosed with COVID-19 who remain at home with mild symptoms will also generate potentially infectious waste. The World Health Organization (WHO) advises that any system that exercises best practices for handling infectious waste must be capable of managing waste potentially infected with SARS-CoV-2. Specifically, waste associated with COVID-19, including PPE (Personal Protective Equipment), is not managed differently from other infectious waste.

PPE is defined as any input, equipment, apparatus, or device that is specially designed and manufactured to protect the human body, in whole or in part, from specific risks of work accidents, occupational diseases, or dangerous environments. Various types of PPE exist for different types of risks, but it generally serves to protect the head, ears, face, eyes, upper limbs, and lower limbs. The official Mexican standards in which criteria are established for the use of PPE in Mexico are: NOM-017-STPS-2008, NOM-113-STPS-2009, NOM-023-STPS-2012, and NOM-026-STPS-2008026, which are regulated by the Ministry of Labor and Social Welfare (Secretaria del Trabajo y Previsión Social).

The use of PPE by healthcare workers and the general public has increased dramatically, so establishments that provide specialized waste management services are overwhelmed. The exact viability of SARS-CoV-2 is currently not known. However, the best scientific evidence available at this time indicates that it may be up to 3 days on hard surfaces such as plastic.

In addition, outside the hospital environment, masks, disposable tissues, face coverings, PPE, and other non-biodegradable waste related to SARS-CoV-2 must be collected separately, deposited in a double bag, and labeled with the date. If there is any possibility that any PPE could be improperly and illegally reused, it must be transformed so as to eliminate its inherent morphological characteristics and thereby render it unrecognizable. Once the PPE has undergone the sterilization and transformation processes, the resulting waste can be sent for final disposal or recycling.

Since PPE is a tool for limiting the risk of contagion in health personnel involved in caring for patients who are suspected of being infected or are diagnosed with COVID-19 as well as in the general population that is potentially exposed to the SARS-CoV-2 virus, these inputs, once used, are classified as Biohazardous Waste (RPBI in Mexico).

Biohazardous wastes are those materials generated during medical care services, at funeral homes, biological companies, inter alia, which contain biohazardous agents and can cause harmful effects to health and the environment. In accordance with Official Mexican STANDARD NOM-087-ECOL-SSA1-2002, biohazardous waste must be treated by physical or chemical methods that ensure the elimination of pathogenic microorganisms and that the processed waste is unrecognizable for final disposal thereof.

The treatment of biohazardous waste is currently carried out using different methods, with the most commonly used being incineration, sterilization by autoclaving, application of microwaves, and finally sterilization or chemical disinfection. Biohazardous waste best management practices indicate that it should be treated using non-incineration methods. On account of the Persistent Organic Pollutants (POPs) produced by incineration, both the WHO (World Health Organization) and the United Nations Environment Program (UNEP) support processes for managing infectious waste that do not employ incineration. Incineration is a much less efficient process than other existing technologies, and it has a greater carbon footprint. Capturing energy from burning waste is the most polluting and expensive form of energy generation. With regard to chemical sterilization, different disinfectants such as chlorine dioxide, sodium hypochlorite, ethylene oxide, formaldehyde gas, glutaraldehyde, or peracetic acid can be used. Peracetic acid has exhibited great biocidal capacity, and the compounds are completely innocuous when released into the environment.

Peracetic acid, also called peroxyacetic acid or simply PAA, is known for its high oxidative capacity. This compound has a condensed formula of CH₃COOOH and a molecular mass of 76.05 g/mol. This aggregate has a pungent odor, is clear, and is normally diluted with water when used as a sterilizing agent. The most common aqueous formulations contain peracetic acid in a concentration of between 5, 15, and 35 percent with respect to the total solution, and it is formulated in the presence of acetic acid and hydrogen peroxide; these two compounds are in chemical equilibrium and form PAA. Concentrations below 5% have been observed to have broad antimicrobial and biocidal activity, and studies exist which demonstrate the elimination of bacteria and fungi. Nevertheless, the percentage characterization that is to be employed and the effect thereof on viruses is still limited.

Due to its oxidizing capacity and rapid action, peracetic acid in dilution with hydrogen peroxide has unique advantages over other disinfectants. The environmental impact of this mixture is non-existent, since it hydrolyzes readily and very quickly (between 20 to 25 minutes at a low concentration in water) into acetic acid and oxygen. At present, this component is used in the food industry for cleaning fruit, vegetables, and meat processing rooms. However, given its great germicidal capacity against virus (spores, bacteria, and fungi), it is used for the sterilization of surgical instruments and virus culture areas in pharmaceuticals, the latter representing an advancement over discarding aldehydes as sterilants.

Finally, PAA offers the possibility of being used for the sterilization of biohazardous waste and combined with a destruction process (leaving them unrecognizable); the biohazardous waste generated by the PPE treated in this manner could be classified as unrecognizable special waste. This would provide an alternative with no environmental impact AND no risk of exposure to SARS-CoV-2 infection for personnel and the general population. Previous Solutions.

According to official Mexican standard NOM-087-ECOL-SSA1-200, the management of biohazardous waste includes different operations ranging from the identification, separation, packaging, stockpiling, collection and transport, treatment, and final disposal of the waste. Likewise, the standard indicates that biohazardous waste must be treated by physical or chemical methods that ensure the elimination of pathogenic microorganisms and transform them into unrecognizable materials. Some solutions have been described in which chemical methods are used to eliminate pathogenic microorganisms from biohazardous waste, but they do not have an integrated process for rendering it unrecognizable; indeed, there is no news of any comprehensive solution that allows for the inactivation of biohazardous waste and the destruction thereof to the point of becoming unrecognizable material while also offering the possibility of recycling.

### PRIOR ART

Based on an analysis of prior art documents, inventions do exist which attempt to solve similar problems, as is the case of the invention described in document MX2011011577A of Oct. 31, 2011, which discloses a mixture in aqueous solution containing formaldehyde, ethylene oxide, and methanol for sterilizing biohazardous waste that is the subject of a management plan to eliminate the infectious characteristics thereof. However, unlike our invention, it does not propose a method for rendering the waste unrecognizable, nor does it present the option of being able to recycle the waste.

The invention detailed in document EP0442909B1, dated Oct. 14, 1988, describes an improved infectious waste treatment system in which infectious waste is broken up and converted into a liquid or liquid/sludge form and then chemically disinfected by means of a disinfectant in a precisely controlled batch process over time. The disinfection process takes place in a hermetically sealed container to ensure against the release of pathogens into the atmosphere. The process produces a non-toxic and non-infectious liquid or liquid/sludge residue that can be safely discharged into a sanitary sewer or otherwise disposed of environmentally. The peracetic acid-based chemical constituting the basis of our invention is not used in this process.

The invention described in document U.S. Pat. No. 6,096,266A of Jul. 10, 1998 describes a method for disinfecting and sterilizing materials contaminated by microbes in which contaminated materials, such as medical instruments and waste from medical laboratories or hospitals, are disinfected and sterilized using a combination of peracetic acid and ozone in a sterilization chamber. Peracetic acid is formed within the sterilization chamber by introducing liquid hydrogen peroxide and liquid acetic acid therein. Unlike our invention, the development of the disinfectant material does not require a solution generation device that includes a device and a formulation.

On the other hand, document EP0945404A2, dated Mar. 23, 1998, describes an invention in which the disinfectants according to the invention with high microbicidal activity contain up to 5% by weight of performic acid in addition to hydrogen peroxide and peracetic acid; these can be obtained by adding a source of formic acid, such as formic acid, a water-soluble salt thereof, or a lower formic acid ester, to an aqueous phase containing 5 to 50% by weight of hydrogen peroxide and 1 to 15% by weight of peracetic acid and, if a salt is added, a mineral acid as well. The preferred agents are obtained through the addition of 0.1 to 1 mol of sodium formate or formic acid per kg of the agent to be produced to an equilibrium peracetic acid containing mineral acid with 0.5 to 6% by weight of peracetic acid. That invention, although it makes use of peracetic acid, contains a different formulation from the one developed herein, so that both the application and the formulation itself are totally different from our invention, which additionally allows for the integration of a process for disposing of the infected items.

The invention described in document U.S. Pat. No. 5,520,888A dated Feb. 1, 1994, where a reactor/sterilizer for disinfecting contaminated medical and/or biological waste comprises, according to the invention, at least one grinder to reduce the size of solid waste that is to be disinfected, means for feeding waste into the grinder, a freezer or ice maker that traps sanitizing concentrations of ozone from an ozone generator in ice so that ice can be added to biomedical waste prior to a shredding step. In the disinfection method, the invention relates to treating biological and/or medical waste with ozone-containing ice by mixing the ice with the waste and allowing the ice to melt, thereby releasing ozone and exposing the waste to effective ozone concentrations in order to disinfect the waste. In preferred embodiments, the biomedical reactor/sterilizer has both a coarse shredder and a grinder. The grinder produces a slush or slurry of waste and ozone that contains ice. As the slurry temperature increases, the trapped ozone is released in a sustained manner as the ice melts over time and mixes with and disinfects the debris. This invention, although it seeks to solve the same problem, differs in that the form and proportions of the formulation described are totally different by virtue of the formulation of our invention, since it has a greater virucidal capacity due to the content of its formulation.

Based on an analysis of prior art, it can be seen that, with respect to the previously described problem of the sterilization of personal protective equipment, there are no inventions that have been developed or tested for cases of exposure to SARS-CoV-2, which is quite new, and the invention described in this document provides the virucidal and disinfectant capacity for hazardous biological waste in the same place in which the infected equipment is generated, since the infectious capacity of the virus is such that it requires immediate and effective actions to avoid spread through personal protective equipment exposed to the virus.

It is the object of the invention to provide a formulation and method for the treatment and final disposal or recycling of PPE that has been potentially contaminated or is totally contaminated by SARS-CoV-2, thereby eliminating the infectious characteristics thereof and transforming it into unrecognizable materials. This formulation and method allows for:
inactivation of the PPE in the place in which it is generated in order to reduce the risk of exposure and contagion to health personnel and the general population,
destruction of the PPE so as to render it unrecognizable in order to prevent reuse and possible contagion,
use a grinding process in combination with a peracetic acid-based formulation for the treatment of:
   PPE contaminated by SARS-CoV-2
   biohazardous wastes,
   municipal waste contaminated by SARS-CoV-2,
      said formulation and method consisting of the combination of the simultaneous use of a peracetic acid formulation and a grinding process. By subjecting the PPE exposed to SARS-CoV-2 to the formulation and method, the resulting reaction formulation substantially increases the virucidal efficacy through the inactivation of the genetic material of the virus and the transformation of the inputs by eliminating the inherent morphological characteristics thereof. This formulation and method simultaneously and efficiently sterilize and transform the inputs in the very place in which they are generated, thus eliminating conventional collection and transportation processes without producing polluting emissions and resulting in sterile waste that is unrecognizable from its original form and ready for final disposal as municipal solid waste or for recycling. The technical details of the developed invention are described below.

### BRIEF DESCRIPTION OF THE FIGURES

FIG. 1 shows the waste destruction process and the parameters used.

FIG. 2 shows the monitoring of cell cultures exposed to supernatants treated with peracetic acid/hydrogen peroxide, negative control (not exposed to supernatant) and positive control (exposed to untreated supernatant). The columns indicate the culture time, and the rows the type of exposure.

FIG. 3 shows the monitoring of MCF7 (Michigan Cancer Foundation 7), negative control (A-O), positive control exposed to the supernatant obtained from the biohazardous waste bags prior to processing (E-H) and post-process leaching (I-H). Arrows indicate cytopathic damage (cell death).

FIG. 4 shows the monitoring of HEPG2 (hepatocellular carcinoma), negative control (A-D), positive control exposed to the supernatant obtained from the biohazardous waste bags prior to processing (E-H), post-process leaching (1-H), and the cells exposed to the solids obtained after the integral grinding and crushing treatment that simultaneously disinfects the biohazardous waste, reducing its volume by up to 90% and rendering it harmless for disposal as ordinary waste. Arrows indicate cytopathic damage (cell death).

FIG. 5 shows the histogram of cell viability based on the result of the MTT assay (cell proliferation assay), which is a method for determining the possible cytotoxic effect of an agent on tumor cell lines or primary cultures of normal cells; the Y axis represents the percentage of viability of each well, and the X axis represents the type of treatment and control. The bars have their standard deviation.

### DETAILED DESCRIPTION OF THE INVENTION

The characteristic details of the formulation and method used for the sterilization, transformation, and final disposal or recycling of personal protective equipment exposed to SARS-CoV-2 in the same place where it is generated are clearly described in the following description and in the appended illustrative drawings.

### Grinding and Crushing Process for Waste Treatment.

A closed biohazardous waste chemical grinding and washing system is recommended for carrying out this process. For this system, a three-phase electrical connection of 230 V at 60 Hz is recommended. Likewise, a cold water intake (at the temperature of the water supply intake), ¾ inch at a pressure of 3.5 to 5.5 bar, should be used. A drain outlet of at least 50 mm diameter or at least 2 inches at ground level is recommended for the wastewater outlet. The lighting can be at a normal level (domestic with natural light or 2 100-watt incandescent bulbs), an internet connection is recommended (since the equipment can be monitored remotely), and it must be installed in a ventilated place capable of providing a free space with a recommended area of 3×4 meters.

For the application of the formulation, a grinding process was developed for the treatment of the waste which includes the combination of the process and the peracetic acid-based formulation that transforms biohazardous waste into unrecognizable special waste. This process consists of grinding and chemical disinfection under controlled conditions for time and the concentration of the chemical sterilization solution, whereupon the volume of the waste is reduced by destroying the waste until it is unrecognizable and pathogens are rendered inactive. As a result of the process, leachate is obtained having characteristics that enable it to be disposed of in general drainage, as well as transformed solid waste that is free of pathogens and can be treated as special waste that can be recycled or disposed of in the manner of municipal garbage. In Mexico, the leachate can be discarded in general drainage in compliance with official Mexican standard NOM-001-SEMARNAT-1996, which establishes the maximum permissible limits of contaminants in wastewater discharges in national waters and lands. These limits are on the concentration of basic contaminants, as well as pathogenic and parasitic contaminants, acute toxicity, and true color, and heavy metals and cyanides. On the other hand, also in the case of Mexico, the transformed solid waste can be treated as special waste in compliance with official Mexican standard NOM-052-SEMARNAT-2005, which establishes the characteristics, the identification, classification, and lists of hazardous waste. This standard indicates that waste shall be considered hazardous if it presents any of the following characteristics: corrosivity, reactivity, explosivity, environmental toxicity, flammability, and bio-infectiousness. It is important to mention that the permissible limits may vary according to the application-specific regulations in a given country.

The waste destruction process consists of the following stages: loading, grinding, chemical washing, drainage, and output of solids. In the loading stage, the biohazardous waste generated as a result of exposure of the personal protective equipment to SARS-CoV-2 is introduced, and the sprinklers carry out a first rinse in the upper chamber of the equipment. During grinding, the waste is destroyed and then exposed to a chemical wash through the action of a mixture of water with peracetic acid. Finally, during drainage and output of solids, the leachate comes out through a drain valve, and the sterilized solids are removed by means of a screw conveyor. This solid waste is collected in containers for its subsequent final destination as common garbage.

### The Specific Grinding and Crushing Method Consists of the Following Steps:

a. A waste destruction process that transforms biohazardous waste generated by exposure to SARS-CoV-2 into unrecognizable special waste, this process being carried out in a 20-minute cycle and with a processing capacity of 10 to 66 kilograms of contaminated waste and comprising the steps:
   i. loading, grinding, and chemical disinfection under controlled conditions for time and the concentration of the chemical sterilization solution;
   ii. reduction in the volume of waste by 80% to 90% by destroying the waste until it is unrecognizable and pathogens are rendered inactive;
   iii. as a result of the process, leachate is obtained with characteristics that are apt for disposal in general drainage in compliance with the permissible limits specified by the applicable regulations of the country in question and unrecognizable solid waste that has been transformed, is free of pathogens, and can be treated as special waste in compliance with the characteristics specified by the applicable standards of the country in question,
b. the waste destruction process consisting of the following stages:
   1. Loading. In this stage, the biohazardous waste weighing between 10 and 66 kg generated through exposure of the personal protective equipment to SARS-CoV-2 is introduced, and the sprinklers carry out a first rinse in the upper chamber of the equipment.
   2. Grinding. In this stage, the destruction of the waste is carried out in a closed system that shreds all of the waste placed in a loading chamber for at least 4 minutes that generates small pieces of garbage, no more than 5 mm long, and a reduction in volume of between 80% to 90%;
   3. Chemical washing. This stage is carried out through the action of a mixture of water with peracetic acid (which corresponds to the formulation developed that is described in the following sections);
   4. Drainage. In this stage, the leachate comes out through a drain valve.
   5. Output of solids. In this stage, the solid waste is collected in containers for its subsequent final destination or possible recycling, as it does not present the characteristics of: corrosivity, reactivity, explosivity, environmental toxicity, flammability, and bio-infectiousness.

FIG. 1 shows the elements of the waste destruction process with the parameters used: 65 kg of waste in yellow bags, 700 ml of peracetic acid, in 10 liters of water for a 20-minute cycle. After the aforementioned stages, 50 liters of leachate is obtained which passes through the drain, as well as 15 kg of pathogen-free and unrecognizable solid waste.

In order to be able to dispose of the biohazardous waste in the manner of municipal garbage, it must not have any pathogenic microorganism that continues to be considered a biohazardous waste. Therefore, between 72,000 and 88,000 parts per million (PPM) or 72 to 88 grams per liter of peracetic acid was obtained upon producing the mixture of hydrogen peroxide, acetic acid, and sulfuric acid. These components are found in the proportions of 40-80% (hydrogen peroxide), 10-40% (acetic acid), and 1-10% (sulfuric acid), with acetic acid (CH₃COOH) and hydrogen peroxide hydrogen (H₂O₂) reacting to form peracetic acid, and with the presence of sulfuric acid (H₂SO₄) stabilizing this molecule. Its stable formula is as follows:

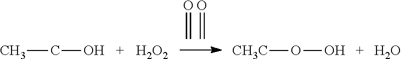

This solution was diluted to 1 and 0.5% volume/volume with potable water. This yielded solutions of 200 to 400 ppm or 200 to 400 mg per liter of peracetic acid. These solutions were used to validate their virucidal capacity in biohazardous wastes contaminated with New Castle-type viruses.

### Cell Culture Monitoring.

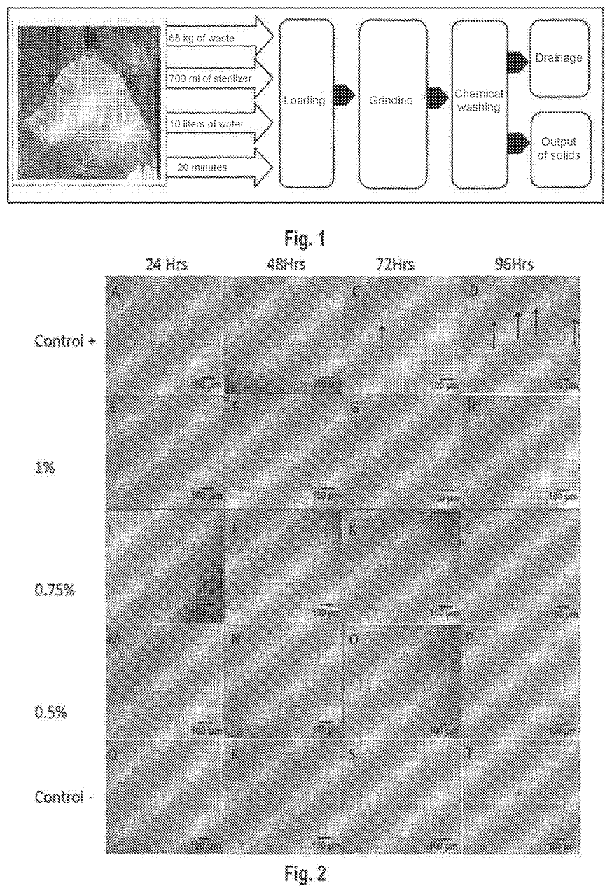

A cell culture of HEK 293 cells (human embryonic kidney 293 cells) distributed in a 24-well plate was monitored for 96 hours. During the first 24 hours after the exposure of the supernatant liquid to the biohazardous waste, untreated and treated with the sterilizing solution, no cytopathic evidence was found, as can be seen in FIGS. 2A, 2G, 2K, 20, 2S. The culture in the different wells of the controls and test samples continued to grow and proliferate as normal; however, at 72 hours the positive control wells that were exposed to the supernatant liquid of the waste without treatment with the sterilizing liquid began to manifest signs of cytopathy, as is shown in FIG. 2D. Finally, at 96 hours, severe cytopathic effects were observed, which are shown in FIG. 2E, whereas the other cultures exhibited normal growth, similar to that of the negative control (non-exposed cells).

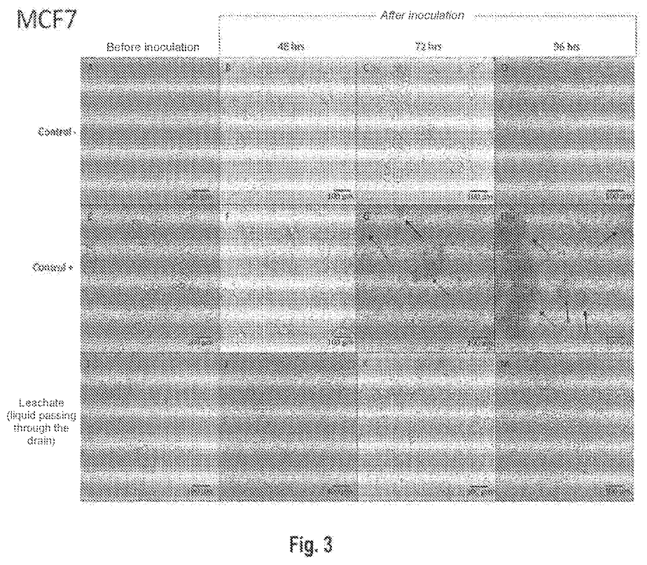

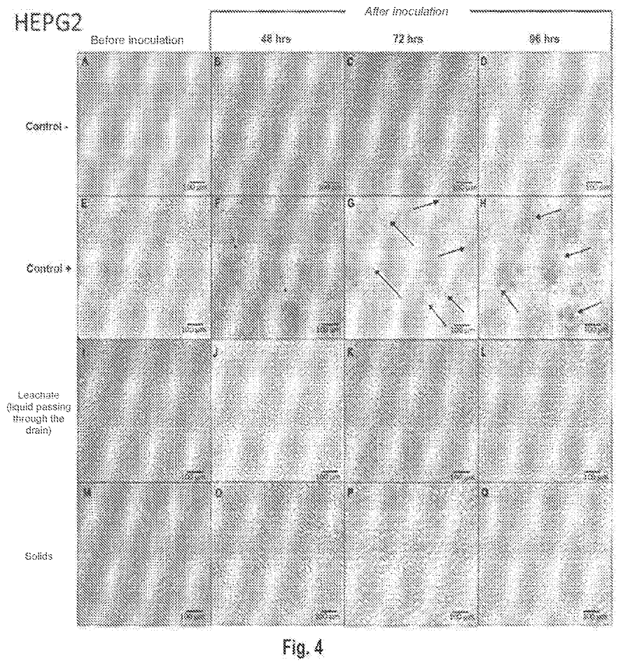

On the other hand, HEPG2 and MCF7 cells exposed to leachate and solids only for HEPG2 exhibit a behavior similar to that of HEK293, with the exposed cultures not showing any signs of cytopathy, just like the negative control; this is shown in FIGS. 3 and 4. However, the cytopathic evidence is clear in the positive controls from 72 and 48 hours, respectively, which can be seen in FIG. 3F and FIG. 4C.

### MTT Assay (Cell Proliferation Assay).

After MTT processing, FIG. 3 shows that the cells that were exposed to the viral supernatant have a viability of 80 to 95% compared to the negative control. They do not mimic the behavior of the negative control, since the neutralization process and the possible salts that are formed might affect the cell culture, inducing cell death due to an osmotic imbalance.

However, the positive control has a viability of 45%, showing cell death due to the presence of the virus and the replication and proliferation thereof. In order to avoid having a low cell viability in the negative control that might affect the reading, the culture was not continued for another 24 hours. MTT was not necessary for HEPG2 and MCF7, since cell death at 96 hours was evident for both cultures in the positive control, whereas in the negative control and in the experimental wells the confluence reached exceeded 90%; this is shown in FIGS. 3D, 3M, and in FIG. 4, corresponding to 4D, 4L, 4Q.

Process Validation and Formulation with Waste with Other Microorganisms.

The bactericidal and fungicidal action was verified using five different species: Escherichia coli and Pseudomona aureginosa, representing the bacteria in the gram-negative group; Staphylococcus aureus and Bacillus subtillus, representing the bacteria in the gram-positive group, and Candida albcans as a fungus. These microorganisms were allowed to grow on nutrient media to proliferation. After 24 hours, 3 batches of waste prepared with sterile residue from needles, syringes, sheets, compresses, and cotton pads were contaminated. Samples were taken from the various proliferating suspensions, with dilutions up to 10×⁻⁶ being prepared in triplicate. Each batch was then eliminated in the system with concentrations of 0.5 and 1% volume/volume for 5 minutes, respectively (washing). At the end of each process, 3 ml of the liquid sample were neutralized with sodium hydroxide. Subsequently, each sample was taken for subsequent culturing in specific agar solutions for each microorganism; these were allowed to incubate for 24 hours. After incubation, the Colony Forming Units (CFU) were counted for quantification.

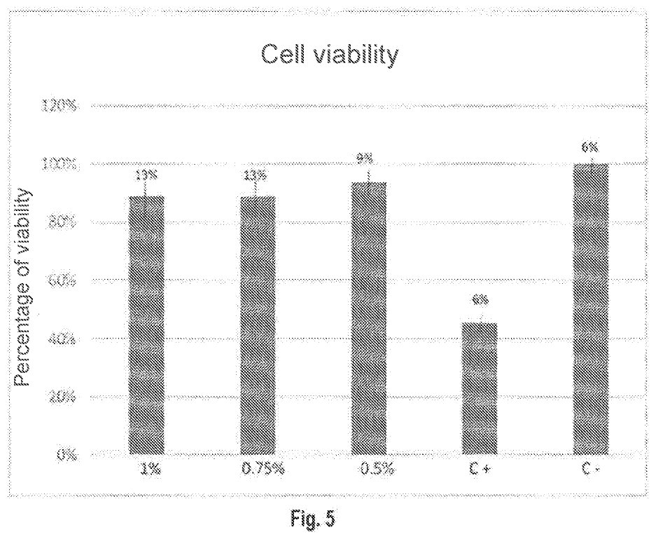

A clear elimination of pathogens from 6 Log₁₀ to 7 Log₁₀ was observed among all species, the cultures having been left exposed to the practical acid solution with no evidence of any colony formation. Formed colonies were observed in the positive controls, indicating the proliferation of the microorganism. FIG. 5 shows the cell viability based on the result of the MTT assay.

The sterilizing liquid and the grinding process show efficacy in the elimination of the New Castle virus from the biohazardous waste, and the absence of other microorganisms that might cause these wastes to be categorized as biohazards was likewise confirmed. The cultures that were inoculated with the treated and neutralized supernatant behaved like a healthy culture, whereas the control that was inoculated with the untreated supernatant fluid exhibited signs of mild (at 72 hours) to severe (94 hours) cytopathy. Likewise, the presence of other microorganisms such as bacteria and fungi was verified, demonstrating that the combination of peracetic acid and a grinding system is a binomial for the transformation of biohazardous waste to special waste which, in addition to being free of pathogens, is unrecognizable.

### Formulation and Resulting Method.

The formulation that was obtained as a result of all of the analyses described and yields the best results is as follows:

A chemical formulation for the treatment and final disposal or recycling of personal protective equipment (PPE) exposed to SARS-CoV-2, characterized in that it comprises:
a. for the elimination of microorganisms in biohazardous waste from exposure of the personal protective equipment to SARS-CoV-2:
   i. peracetic acid at 23% weight/weight in water, which is obtained by mixing hydrogen peroxide, acetic acid, and sulfuric acid;
   ii. the proportions of the above components in numeral i are:
      1. hydrogen peroxide 40-80%,
      2. acetic acid 10-40%, and
      3. sulfuric acid 1-10%;
   iii. wherein acetic acid (CH₃COOH) and hydrogen peroxide (H₂O₂) react to form peracetic acid, with the presence of sulfuric acid (H₂SO₄) stabilizing this molecule;
   iv. the solution is diluted in 1% hydrogen peroxide, 0.75% acetic acid, and 0.5% sulfuric acid volume/volume with potable water;
   v. all of the above yields solutions of 200 to 400 ppm or the equivalent thereof of 200 to 400 mg per liter of peracetic acid.

In addition, and in conjunction with the formulation described, a crushing and grinding process is required in order to render biohazardous waste from exposure of personal protective equipment to SARS-CoV-2 unrecognizable with the option of being recycled, and it provides the appropriate physical environment for the reaction mixture with the peracetic acid-based chemical agent of the resulting formulation, said process being characterized in that it comprises:
a. a waste destruction process that transforms biohazardous waste generated by exposure to SARS-CoV-2 into unrecognizable special waste, said process comprising the steps:
   i. grinding and chemical disinfection under controlled conditions for time and the concentration of the chemical sterilization solution;
   ii. reduction in the volume of waste by destroying the waste until it is unrecognizable and pathogens are rendered inactive;
   iii. as a result of the process, leachate is obtained having characteristics that enable it to be disposed of in general drainage, as well as unrecognizable transformed solid waste that is free of pathogens and can be treated as special waste;
b. the waste destruction process consisting of the following stages:
   1. Loading. In this stage, the biohazardous waste generated through exposure of the personal protective equipment to SARS-CoV-2 is introduced, and the sprinklers carry out a first rinse in the upper chamber of the equipment.
   2. Grinding. In this stage, the destruction of the waste is carried out.
   3. Chemical washing. This stage is carried out through the action of a mixture of water with peracetic acid.
   4. Drainage. In this stage, the leachate comes out through a drain valve.
   5. Output of solids. In this stage, the solid waste is collected in containers for its subsequent final destination or possible recycling.

The reaction mixture for the treatment and final disposal or recycling of PPE contaminated or potentially contaminated by SARS-CoV-2, eliminating the infectious characteristics thereof and transforming it into unrecognizable materials. This reaction mixture allows for:
the inactivation of the PPE in the place in which it is generated in order to reduce the risk of exposure and contagion to health personnel and the general population,
destruction of the PPE so as to render it unrecognizable in order to prevent reuse and possible contagion,
the use of a grinding process in combination with a peracetic acid-based formulation for the treatment of:
   PPE contaminated by SARS-CoV-2,
   biohazardous wastes,
   municipal waste contaminated by SARS-CoV-2.

The preceding description of the disclosed definitions is provided in order to enable any person skilled in the art to implement or use the present invention. Various modifications to these definitions and/or implementations will be readily apparent to those skilled in the art, and the generic principles defined herein may be applied to other embodiments without departing from the spirit or scope of the invention. Therefore, the present invention is not intended to be limited to the embodiments shown herein, but should be granted the broadest scope consistent with the following claims and the principles and novel features disclosed herein.

Having sufficiently described my invention, I consider novel and therefore claim as my exclusive property that which is contained in the following clauses:

1. A formulation used for the sterilization, transformation, and final disposal or recycling of personal protective equipment exposed to SARS-CoV-2 with bactericidal, fungicidal, and virucidal action, characterized in that it comprises:
i. peracetic acid at 23% weight/weight in water, which is obtained by mixing hydrogen peroxide, acetic acid, and sulfuric acid;
ii. the proportions of the components of numeral i are:
1. hydrogen peroxide 40-80%,
2. acetic acid 10-40%, and
3. sulfuric acid 1-10%;
iii. wherein acetic acid (CH₃COOH) and hydrogen peroxide (H₂O₂) react to form peracetic acid, with the presence of sulfuric acid (H₂SO₄) stabilizing this molecule;
iv. the solution is diluted in 1% hydrogen peroxide, 0.75% acetic acid, and 0.5% sulfuric acid volume/volume with potable water;
v. all of the above yields solutions of 200 to 400 ppm or the equivalent thereof of 200 to 400 mg per liter of peracetic acid.

2. A method for the sterilization, transformation, and final disposal or recycling of personal protective equipment exposed to SARS-CoV-2 from biohazardous waste and that provides the appropriate physical environment for a reaction mixture with a peracetic acid-based chemical agent, characterized in that it comprises:
a. a grinding and crushing process, comprising:
i. a waste destruction process that transforms biohazardous waste generated by exposure to SARS CoV-2 into unrecognizable special waste, this process being carried out in a 20-minute cycle and with a processing capacity of 10 to 66 kilograms of contaminated waste and consisting of:
1. loading, grinding, and chemical disinfection under controlled conditions for time and the concentration of the chemical sterilization solution;
2. reduction in the volume of waste by 80% to 90% by destroying the waste until it is unrecognizable and pathogens are rendered inactive;
3. as a result of the process, leachate is obtained with characteristics that are apt for disposal in general drainage.
ii. The waste destruction process consists of the following stages:
1. Loading. In this stage, the biohazardous waste weighing between 10 and 66 kg generated through exposure of the personal protective equipment to SARS-CoV-2 is introduced, and the sprinklers carry out a first rinse in the upper chamber of the equipment.
2. Grinding. In this stage, the destruction of the waste is carried out in a closed system that shreds all of the waste placed in a loading chamber for at least 4 minutes that generates small pieces of garbage, no more than 5 mm long, and a reduction in volume of between 80% to 90%;
3. Chemical washing. This stage is carried out through the action of a mixture of water with peracetic acid containing solutions of 200 to 400 ppm or the equivalent thereof of 200 to 400 mg per liter of peracetic acid.
4. Drainage. In this stage, the leachate comes out through a drain valve.
5. Output of solids. In this stage, the solid waste is collected in containers for its subsequent final destination or possible recycling, as it does not present the characteristics of: corrosivity, reactivity, explosivity, environmental toxicity, flammability, and bio-infectiousness.
